(19) United States
(12) Patent Application Publication
(10) Pub. No.: US 2015/0202087 A1
(43) Pub. Date: Jul. 23, 2015

(54) GOGGLE

(71) Applicant: ASWAN INTERNATIONAL CORP., TAIPEI CITY (TW)

(72) Inventor: YUNG-AN CHEN, TAIPEI CITY (TW)

(73) Assignee: ASWAN INTERNATIONAL CORP., TAIPEI CITY (TW)

(21) Appl. No.: 14/160,842

(22) Filed: Jan. 22, 2014

(51) Int. Cl. A61F 9/02

(52) U.S. Cl. CPC A61F 9/026

(57) ABSTRACT

A goggle includes a frame a soft skirt. The soft skirt is disposed at a side of the frame and has an adjustment portion having a plurality of teeth arranged in intervals. The goggle conforms to a wearer's nose and the skin around.

### BACKGROUND

1. Field of the Invention

The instant disclosure relates to an eyewear; in particular, to a goggle for eye protection.

2. Description of Related Art

Eyes are relatively fragile compared to the other parts of human body, and therefore during skiing, swimming and other exercising or some working environment, safety goggles are needed to protect the eyes. The goggles prevent foreign particles enter the eyes. There is an urgent need to provide reliable and comfortable goggles that conform to the eye region.

Conventional goggle, as shown in FIG. 1, includes a frame 1A, a lens 2A and a skirt 3A. The lens 2A is mounted on the outer side of the frame 1A. The frame 1A is formed according to the contour of the nose bridge as an inverted V. The skirt 3A is formed along the inner rim of the frame 1A and is flat. When putting on the goggle, the skirt 3A conforms to the eye region, and a band is provided to adjust the eyewear to suitable tightness. However, the inverted V of the frame 1A is preset, and it is unlikely to fit each and every wearer. Therefore, the conventional eyewear heavily relies on the band to tighten about a face. The skirt 3A is pushed to attach to the skin of the wearer. Nevertheless, more pressure amounts to the face, and the flat skirt 3A can hardly seal to a wearer's face. As a result, a gap is formed between the skirt 3A and the wearer's face, and the goggle can easily slip off. The goggle has to be constantly adjusted which causes inconvenience.

To address the above issues, the inventor strives via associated experience and research to present the instant disclosure, which can effectively improve the limitation described above.

### BRIEF SUMMARY OF THE INVENTION

The instant disclosure provides a goggle having soft skirt which conforms to any sorts of facial contour and nose.

According to one embodiment of the instant disclosure, the goggle includes a frame a soft skirt. The soft skirt is disposed at a side of the frame and has an adjustment portion having a plurality of teeth arranged in intervals.

Human nose features vary widely from one person to another. Because the soft skirt includes the spaced teeth, when wearing, each of the teeth may be pushed and then tilted or deformed. Furthermore, each of the teeth are spaced apart such that one tooth does not affect the neighboring teeth. That is to say, the goggle can undergo subtle angle to accurately fit a wearer's face. In general, the goggle of the instant disclosure can fit to any sorts of face without the worry of loosening. The soft skirt is relatively more comfortable compared to the conventional goggle having flat pad.

In order to further understand the instant disclosure, the following embodiments are provided along with illustrations to facilitate the appreciation of the instant disclosure; however, the appended drawings are merely provided for reference and illustration, without any intention to be used for limiting the scope of the instant disclosure.

### BRIEF DESCRIPTION OF THE SEVERAL VIEWS OF THE DRAWINGS

FIG. 1 is a schematic diagram of a conventional goggle;

FIG. 2 is an exploded view of a goggle in accordance with a first embodiment of the instant disclosure;

FIG. 3 is a perspective view of a goggle in accordance with a first embodiment of the instant disclosure;

FIG. 4-5 are perspective views of a goggle in accordance with a first embodiment of the instant disclosure;

FIG. 6 is a perspective view of a goggle in accordance with a second embodiment of the instant disclosure.

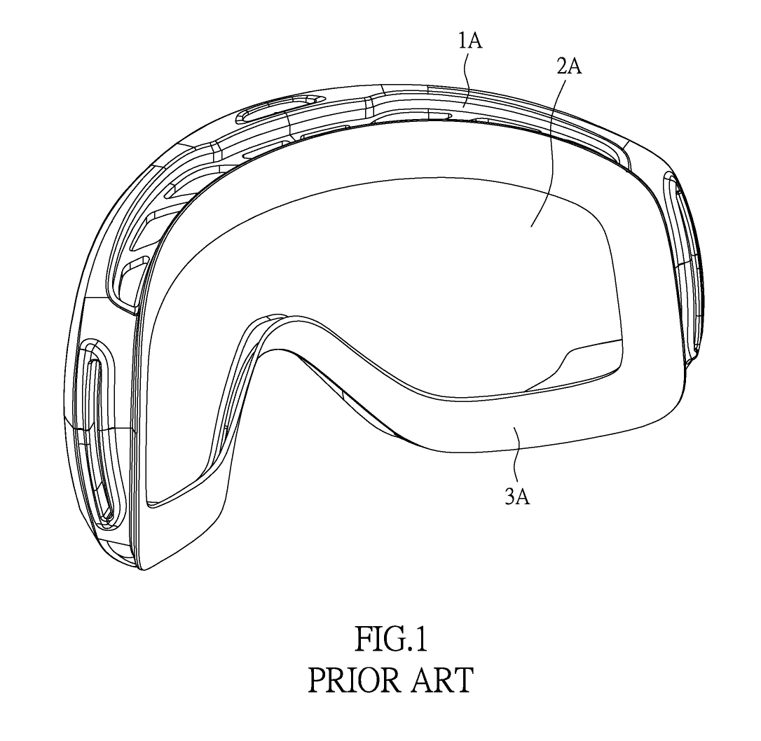

### DETAILED DESCRIPTION OF THE INVENTION

The aforementioned illustrations and following detailed descriptions are exemplary for the purpose of further explaining the scope of the instant disclosure. Other objectives and advantages related to the instant disclosure will be illustrated in the subsequent descriptions and appended drawings.

### First Embodiment

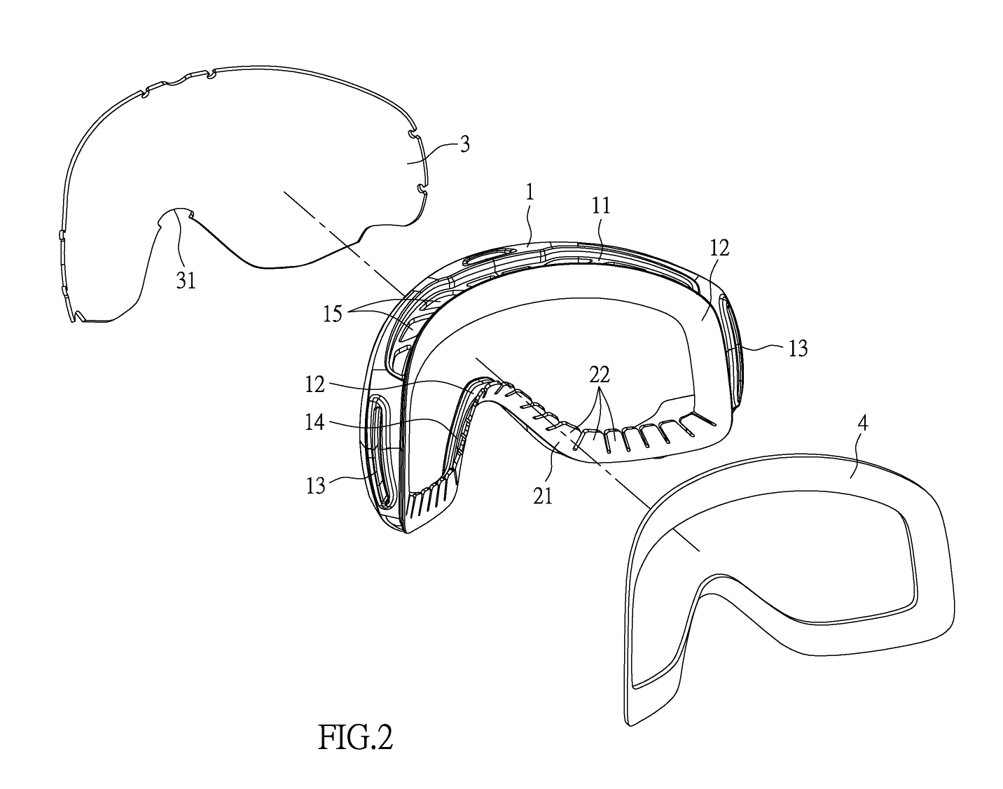

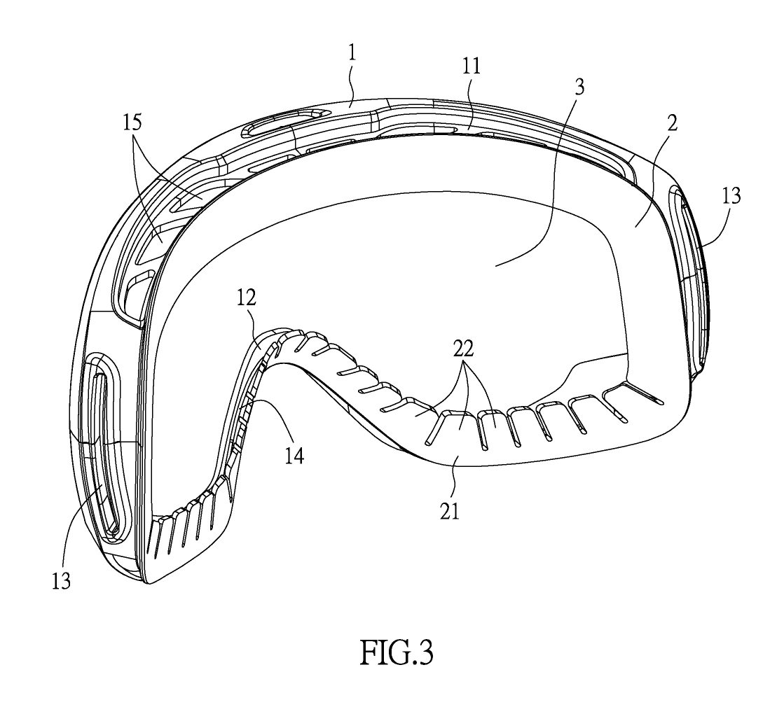

Please refer to FIGS. 2 and 3 showing an exploded view and a perspective view of a goggle in accordance with a first embodiment of the instant disclosure. The goggle includes a frame 1, a soft skirt 2 and a lens 3. The frame 1 has an inner side and an outer side and joining side connecting the inner and outer sides. The soft skirt 2 is formed on an inner side of the frame 1, while the lens 3 is mounted on an outer side of the frame 1 opposite to the soft skirt 2.

Specifically, the frame 1 is defined as an upper portion 11 and a lower portion 12 extending from the upper portion 11. The frame 1 also has two connection portions 13. The frame 1 may be made of polycarbonate (PC), polyethylene (PE), polypropylene (PP), poly(vinyl chloride) (PVC) or other high molecule plastic materials, and the instant disclosure is not limited thereto. The frame 1 is light and robust because of its material. In the instant embodiment, the lower portion 12 conforms to two sides of a nose and across the nose bridge. The two connection portions 13 are depressed on the frame 1 and disposed on the joining side opposite to each other. A band (not shown) can go through the connection portions 13 to secure the goggle.

In another embodiment, the upper portion 11 of the frame 1 is formed with a plurality of holes 15. The sealed space within the frame 1 is then in air communication with the exterior through the holes 15. As a result, air exchange between the space defined by the frame 1 and the exterior. The heat can be exhausted from the holes 15. The number and dimension of the hole 15 are not limited to that as illustrated or described herein and are subject to variation.

The abovementioned band can be elastic. Two ends of the elastic band are coupled to the connection portions 13 respectively, such that the frame 1 is secured to a wearer's face. The shape and material of the band are not limited. The band may be conventional length adjustable strap which allows tightening and loosening. The combining method between the band and the frame 1 is not limited. For example, the band and connection portions 13 may be a pair of male and female latch that mate each other. Alternatively, the band may be coupled to the frame 1 through any method known to a person ordinary skill in the art.

The soft skirt 2 is disposed on the inner rim of the frame 1. The soft skirt 2 may be made of soft, resilient material or deformable high molecule plastic, for example, polybutylene terephthalate (PBT), polypropylene (PP), nylon, styrene butadiene block copolymer (SBC), or poly(styrene-butadiene-styrene) (SBS), and the instant disclosure is not limited thereto. Furthermore, the soft skirt 2 has an adjustment portion 21 disposed along the lower portion 12 of the frame 1.

In the instant embodiment, the adjustment portion 21 has a plurality of teeth arranged in intervals. The teeth are linked at the bottom and attached to the lower portion 12, such that the adjustment portion 21 and the lower portion 12 collectively define a groove 14 for receiving lens 3 when mounted to the frame 1.

The lens 3 is formed with a notch 31 at the central region. More specifically, in the instant embodiment, when the lens 3 is mounted to the frame 1, the top rim of the lens 3 abuts the upper portion 11 of the frame 1, while the bottom rim of the lens 3 is received by the groove 14. Moreover, the notch 31 conforms to an arc at the central region of the lower portion 12, such that the lens 3 is firmly secured in the frame 1.

In anther embodiment, the goggle further includes a pad 4 covers the soft skirt 2. The pad 4 may be made of sponge or other soft materials. If a wearer's skin is more sensitive or wounded, the pad 4 prevents direct contact between the soft skirt 2 and the skin, and therefore the skin will not be irritated by the soft skirt 2. Furthermore, when the goggle is crashed, the pad 4 absorbs the force. Further still, the pad 4 prevents any press mark caused by the soft skirt 2 when wearing for a long period.

When using the goggle, the band is put around the head, and the frame 1 is adjusted to an appropriate position, such that the upper portion 11 and the lower portion 12 of the frame 1 are attached to the forehead, nose and two sides of the nose respectively. The goggle covers the surrounding area of eyes to prevent foreign particles from entering.

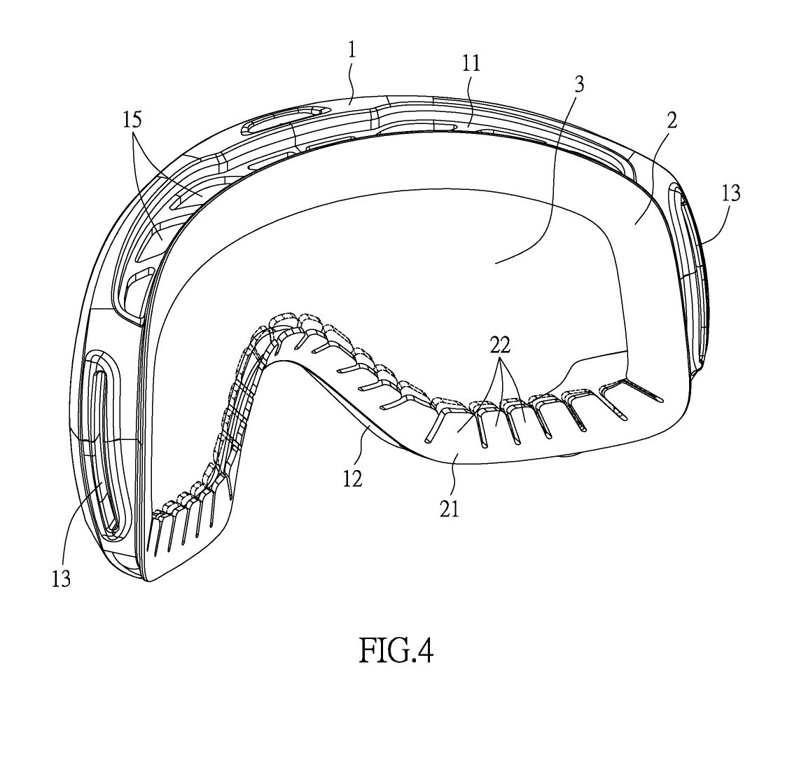

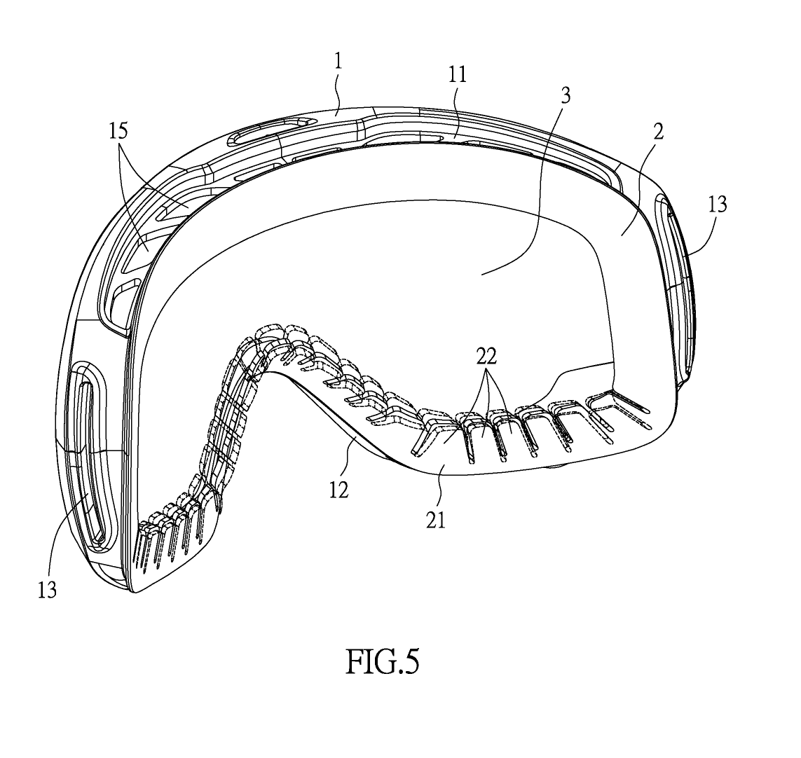

Please refer to FIGS. 4 and 5 showing schematic diagrams of another embodiment. In the instant embodiment, the adjustment portion 21 of the soft skirt 2 has a plurality of teeth 22 arranged in intervals. The teeth 22 facilitate the soft skirt 2 to taper according to the curvature of the nose. More specifically, as shown in FIG. 4, if a wearer has higher cheek bone and distinct nose feature, the teeth 22 corresponding to these regions will tilt and shift slightly toward the lens 3 (as shown in the dashed line). Because each of the teeth 22 is spaced apart, a movement of one tooth 22 does not easily cause the neighboring teeth 22 to shift together. Therefore, each tooth 22 moves subtly according to the curvature of the nose and cheek bone, and the soft skirt 2 is more accurately attached to the nose and two side of the nose.

In addition, as shown in FIG. 5, if a wearer's face is smaller, and the soft skirt 2 cannot abut their face, the adjustment portion 21 can be flicked away from the lens 3 before putting on, such that the adjustment portion 21 abuts the contour of the wearer's face. The adjustment portion 21 conforms to the wearer's face and is adjustable to any angle. Accordingly, the teeth are tilted relative to the lower portion 12 to support the wearer's face, and the soft skirt 2 is more secured on the wearer's face with firm grip.

In short, compared to the conventional goggle, the teeth formed on the soft skirt 2 is capable of adapting any angle, which the flat skirt cannot achieve. The conventional goggle relies on the band to tighten the goggles, and the flat skirt often loosens. The goggle of the instant disclosure is much more comfortable.

In another embodiment, the adjustment portion 21 may be multi-layered to meet different requirement. For example, when the adjustment portion 21 is single-layered, external force may results in the teeth 22 over tilting. The wearer may feel very uncomfortable or even be hurt when the hard frame 1 abuts the face. Therefore, an additional adjustment portion (not shown) may be disposed in the groove 14, such that the adjustment portions cushion between the wearer's face and the frame 1. In addition, the additional adjustment portion reduces the impact when the lens 3 encounters external force and pressures on the wearer's eyes. The additional adjustment portion may also have the plurality teeth arranged in intervals.

### Second Embodiment

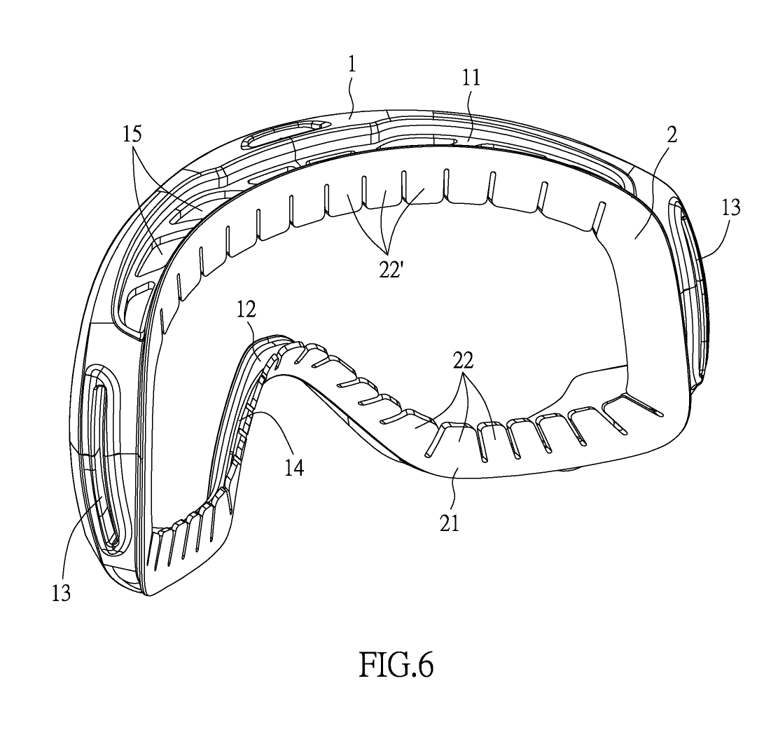

Please refer to FIG. 6 showing a perspective view of the goggle in accordance with a second embodiment of the instant disclosure. In the instant embodiment, the soft skirt 2 close to the upper portion 11 of the frame 1 also has the plurality of teeth 22′ arranged in intervals. The function and other elements are identical as the first embodiment, and it is not repeated herein.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A goggle comprising:
a frame; and
a soft skirt disposed at a side of the frame, wherein the soft skirt has an adjustment portion having a plurality of teeth arranged in intervals.

2. The goggle according to claim 1, wherein the frame has an upper portion and a lower portion conforming to a nose of a wearer, a bottom of the adjustment portion is connected to the lower portion of the frame, and a groove is formed between the adjustment portion and the lower portion.

3. The goggle according to claim 2 further comprising a lens, wherein the lens is disposed on the frame at the other side of the soft skirt, and a bottom of the lens abuts the groove.

4. The goggle according to claim 2, wherein the soft skirt on the upper portion is formed by a plurality of teeth arranged in intervals.

5. The goggle according to claim 2, wherein the upper portion of the frame is formed with a plurality of holes.

6. The goggle according to claim 1, wherein each of the teeth slants toward a direction opposite to the frame to support the wearer's face.

7. The goggle according to claim 1, wherein the adjustment portion is multilayered, and each layer of the adjustment portion has the plurality of teeth arranged in intervals.

8. The goggle according to claim 1 further comprising a pad covering the soft skirt.

9. The goggle according to claim 1, wherein each of two opposite sides of the frame has a connection portion, the goggle includes a band, each end of the band is connected to the connection portion.
